(19) United States
(12) Patent Application Publication
(10) Pub. No.: US 2018/0312893 A1
(43) Pub. Date: Nov. 1, 2018

(54) DIRECT INOCULATION

(71) Applicant: NOVOZYMES A/S, Bagsvaerd (DK)

(72) Inventors: Stuart Michael Stocks, Bagsvaerd (DK); Linda Lehmann, Bagsvaerd (DK); Mads Orla Albaek, Bagsvaerd (DK)

(73) Assignee: NOVOZYMES A/S, Bagsvaerd (DK)

(21) Appl. No.: 15/770,327

(22) PCT Filed: Oct. 20, 2016
(86) PCT No.: PCT/EP2016/075144

§ 371 Date: Apr. 23, 2018

(30) Foreign Application Priority Data
Oct. 21, 2015 (EP) 15190900.9

(51) Int. Cl. C12P 21/00; C12N 9/24

(52) U.S. Cl. CPC C12P 21/00; C12Y 302/01037; C12N 9/248

(57) ABSTRACT

Disclosed is a method for fermenting a microorganism for producing a protein product where seed tanks are omitted with the result that the process is much shorter providing additional flexibility in operating the fermentation plant.

### FIELD OF THE INVENTION

The present invention relates to fermentation technology.

### BACKGROUND OF THE INVENTION

Fermentation processes are well known in the art and many different fermentation protocols can be found in the literature.

A fermentation process is typically divided in the following steps: (a) medium preparation for the growth and production of the process cell at the stages of inoculum development and main fermentation; (b) medium sterilization as well as all the ancillary equipment to assure an aseptic environment; (c) inoculum or seed production which is a pure culture in a sufficient quantity to inoculate the production fermenter; (d) production stage which is conducted in the main fermenter for the product formation; (e) downstream processes to separate and purify the fermentation product; and, (f) treatment and disposal of effluents produced by the process. All these steps are interrelated and the success of the fermentation process depends on an adequate optimization that should be done during the development of the process.

Microorganisms are in general assumed to grow in a number of phases in fermentation, starting with a lag phase where the microorganism is adapting to the medium and start growing, an exponential phase where the microorganism grow at a constant growth rate giving an exponential increase in cell number and cell mass, a stationary phase, where the growth has stopped and the cell number remains constant and finally the death phase where the cell number decreases due to cell death.

The Submerged fermentation process is a common fermentation system and may be of any known set-up, such as a batch process, a fed-batch process or a continuous fermentation process.

A batch fermentation is a process where the growth medium is provided in the fermenter from the start, where the fermenter is inoculated with an intended microorganism and the fermentation process is running until a predetermined condition has been reached, typically depletion of the growth medium and the cessation of microbial growth caused by the depletion.

A fed-batch process is a fermentation where a part of the growth medium is provided from the start of the fermentation process where the inoculum is added, and at a certain time point after the start of the fermentation additional substrate, feed is fed to the fermenter at a rate that may be predetermined or determined by the conditions in the fermenter; until the maximal volume has been reached. The feed may or may not have the same composition as the initial growth medium.

A continuous fermentation process is a process where new growth medium is continuously fed to the fermenter and ferment is simultaneously removed from the fermenter at the same rate so the volume in the fermenter is constant.

In industrial fermentation processes are typically conducted by first providing a growth medium in a fermenter, inoculating the fermenter with an inoculum comprising a microorganism and fermenting under defined conditions such as pH, temperature, oxygen level etc., in a predefined time or until a predefined condition, e.g. titer, oxygen consumption; has been reached.

The inoculum is in general a liquid culture of the microorganism used for the fermentation prepared in a seed fermenter, a fermenter typically having a volume of 5-10% of the main fermenter used for production. The growth medium for the seed fermenter may or may not be the same growth medium as used in the main fermenter.

The seed fermenter is again inoculated with an inoculum having a volume of 5-10% of the volume of the growth medium in the seed fermenter.

Thus the inoculum is typically prepared from a vial containing the production strain, where the vial first is inoculated in a small volume and grown to a desired density to prepare a first culture of the production strain, where after the first production strain is inoculated in the next of a series of fermenters of increasing size, where the volume increases 5-20 fold in each step until a sufficient volume to inoculate the production fermenter has been reached. Such a series of fermenters in increasing size is also known as a seed train.

The inoculum is in general a liquid culture of the microorganism used for the fermentation prepared in a seed fermenter, a fermenter typically having a volume of 5-10% of the main fermenter used for production. The growth medium for the seed fermenter may or may not be the same growth medium as used in the main fermenter. The process of inoculum development involves the preparation of a population of microorganisms from a dormant stock culture (agar plate or vial) to a state useful for inoculating a final productive stage.

### SUMMARY OF THE INVENTION

The invention provides a method for fermenting a microorganism producing a protein product, comprising inoculating a fermenter with said microorganism, wherein the inoculation is done directly with the microorganism without using a seed fermenter.

### BRIEF DESCRIPTION OF DRAWINGS

FIG. 1 shows the total protein content obtained in fermentation broths as described in example 1-5.

FIG. 2 shows the beta-xylosidase activities obtained in fermentation broths as described in example 1-5.

FIG. 3 shows the total protein content obtained in fermentation broths as described in example 6,

FIG. 4 shows the beta-xylosidase activities obtained in fermentation broths as described in example 6.

### DETAILED DESCRIPTION OF THE INVENTION

The invention relates to a method for fermenting a microorganism producing a fermentation product, preferably a protein product, comprising inoculating a main fermenter with said microorganism, wherein the inoculation is done directly, i.e. the inoculum of the microorganism is added to the main fermenter without using a preculture, or a seed train.

The term “main fermenter” is in this description and claims used for the final fermenter used in a fermentation process for producing a fermentation process, wherein the intended fermentation product is produced.

The term “preculture” is understood as a liquid actively growing culture of the microorganism used for inoculating the main fermenter. Actively growing is intended to mean that the culture is in a stage where the microorganism is increasing the number of cells. Thus, the preculture is typically in exponential phase or in late exponential phase where the cells are growing actively. The preculture is in general used as inoculation material in order to avoid or reduce the lag phase in the main fermenter.

The term “seed fermenter” is intended to mean a fermenter wherein the preculture is formed by fermenting the microorganism until a sufficient high cell number for inoculation into the main fermenter.

The term “seed train” is intended to mean a series of seed fermenters of increasing size where the preculture is generated in a series of fermenters of still increasing size where the last fermenter in the seed train has a sufficient size to contain the necessary inoculum for the main fermenter.

The term “inoculum” is intended to mean an amount of the microorganism that is added to the main fermenter in order to start the fermentation process. In case of a fermentation process using seed fermenter the inoculum is typically an amount of the preculture corresponding to 5 to 20% of the volume of the main fermenter.

According to the invention no preculture is used for inoculating the main fermenter, but instead the main fermenter is inoculated using an inoculum of the microorganism in stationary phase or in a dormant stage.

The microorganism in stationary phase may be a liquid culture of a microorganism that has grown until cell division has stopped, or is may be in form of one or more agar plate where the microorganism has grown.

Microorganism growing on an agarplate will grow in several phases at the same time. When the microorganism is inoculated it starts growing forming colonies on the plate and will at the edge of the colonies be in an exponential phase while the microorganisms at the centre of the colonies will be in a stationary or death phase depending on the ages of the plate. Thus when the plate is relatively young the majority of the cells will be in the exponential growth phase, when the plate is more mature the majority of the cells will be in the stationary phase and for old plates the majority of the microorganisms will be in the death phase. The present invention is not dependent on using agarplates comprising microorganisms in a particular phase, however, it is preferred to use a mature agarplate in order to inoculate the fermenter with sufficient amount of the microorganism. According to the invention, an agarplate with a grown microorganism is not considered to be a culture in exponential phase but it is considered as a culture comprising microorganisms in several phases including both exponential, stationary and death phases.

In case that the intended microorganism for a fermentation is a bacterium it should typically grow in one to two days on the agarplate before inoculating the fermenter, if the intended microorganism for a fermentation is a yeast or a fungus is should typically grow for 3-7 days on the agarplate before inoculating the fermenter.

Microorganisms in dormant stage is according to the invention understood as microorganism that have been prepared for long term storage such as frozen or dried cells, such as freeze dried cells. Methods for long term storage are well known in the art and the invention is not limited by the particular selected technique for long term storage.

One preferred inoculum is cell from one or more agarplates or one or more vials comprising frozen cells.

The invention is based on the finding of the inventors that by inoculating the main fermenter using a microorganism in stationary phase or in a dormant stage, such as freeze dried or frozen cells, or cells grown on one or more agar plates where the microorganism has grown instead of using a preculture prepared in a seed fermenter leads to production of essentially same yield of the intended fermentation product in a time that is considerably shorter in comparison with a traditional fermentation process using same conditions except for that the inoculum is in form of a preculture prepared in a seed fermenter.

It is understood that the total time for the production process should be calculated as the time from the microorganism used for fermentation is removed from a long term storable form and transferred to a medium allowing the microorganism to grow, until the time when the main fermentation is completed and recovery, purification, formulation of the fermentation product can begin.

The fermentation method according to the invention may provide a main fermentation that achieves equivalent titre of the intended product in the same time as the time for the main fermentation in a traditional fermentation process using a preculture and seed tank or a seed train. In other embodiments the fermentation method of the invention may provide a main fermentation that achieve equivalent titre of the intended product in a time that is up to 50% longer than the time for the main fermentation in a traditional fermentation process using a preculture and seed tank, such as up to 40% longer, such as up to 30% longer, such as up to 20% longer, such as up to 10% longer but so that the total time for the production process is shorted that the comparable process using preculture prepared in a seed fermenter.

It is known in the art that fermentation processes vary slightly from one batch to another. In this connection the titre of a first fermentation process is considered to be equivalent to the titre of second fermentation process if the titre of first fermentation process falls within the range of activities obtained by repeatedly performing the second fermentation process.

It is rather surprising that not including any seed tank at all results in large scale processes which are then shorter than would be anticipated by conventional thinking, and even more surprising that these processes can actually deliver product concentrations and productivity that is equivalent to process where a traditional preculture is utilised. The conventional thinking is evident in the academic literature surrounding the subject, used in courses around the world, as well as the lore in industry.

The use of “seed tanks” can be seen in the brewing industry, and in the context of modern biotechnology, seed tanks were also considered an essential part of the acetone butanol ethanol (ABE) process developed in the first world war in the UK, the construction of these seed tanks competing for resources in a time of war surely indicates their importance (Hastings, J. J. (1978). Economic Microbiology. In A. H. Rose (Ed.), (pp. 31-45). Academic Press, London.). Useful text books on fermentation technology first appear in 1969 (Solomons G. L., (1969), Materials and Methods in Fermentation. London and New York: Academic Press), where fermentation equipment developed over the previous few decades was first usefully characterized and described for dissemination among practitioners. In this book, no attention is paid at all to whether or not a seed tank must be included, as the assumption of a seed culture is already the practice, and consideration is made only of how to technically achieve the aseptic transfer of materials from one vessel to another. One of the biggest collections of industrial practice and considerations in biotechnology devotes 3 of its 1271 pages to “Inoculum preparation” (Atkinson, B., & Mavituna, F. (1991). Biochemical Engineering and Biotechnology Handbook (Vol. 2nd). New York: Macmillan Publishers Ltd), giving the advice that inocula should be between 3 and 10% for bacterial fermentations, thus a production vessel starting with 100 m³ would justify a 3-10 m³ seed tank, in turn seeded with a 300 to 1000 Litre fermenter, seeded by a 30 to 100 Litre fermenter and so on. Later, in another well known text book (Stanbury P. F., Whittaker, A., & Hall, S. J. (1995). Principles of fermentation technology (Vol. 2). Butterworth-Heinemann), an entire chapter is devoted to “The Development of Inocula for Industrial Fermentations” (Chapter 6), and it is written entirely, from the very first paragraph, with the assumption that a seed tank is required, with the chapter dealing with the development of the seed tank medium and selection of appropriate criteria for transfer of the seed tank to the main tank, and how to minimise “lag phase” in the main tank. The omission of a seed tank is so counter intuitive that no attention is paid whatsoever to its omission, nor is there any consideration that a long lag phase may in fact result in a higher final productivity than a short lag phase—for whatever reason. Another text (Doran, P. M. (1995). Bioprocess Engineering Principles. Academic Press, London & San Diego), implies in its introduction, that the seed process for industrial scale operation is involving three seed stages, a shake flask, a bench-top bioreactor and a pilot-scale bioreactor. Later, another book includes a section on seed stages D. M., Melville, J. C., & Fischer, M. (1999). Optimization of fermentation processes by quantitative analysis: From biochemistry to chemical engineering. In E. M. T. El-Mansi & C. F. A. Bryce (Eds.), Fermentation Microbiology and Biotechnology. London and Philadelphia. Section 6.3.2.), where it also assumes from the very beginning that a seed tank including a liquid medium is a given, and indispensable part of a process, and also assumes that rapid growth in the production vessel is always correlated to higher productivities. Further evidence for the universally adopted assumption to the need for seed stages is the use of the term “fermentation seed train” is used to describe the successively larger seed tanks considered necessary to initiate a commercially useful production fermentation, three liquid seed stages of increasing volume are presented as necessary before a final industrial scale tank can be started (Aehle, W. (2008). General production methods. In Enzymes in Industry. John Wiley & Sons). There are almost certainly many more examples, but the last to be given here is the example in the NREL report (Humbird, D., Davis, R., Tao, L., Kinchin, C., Hsu, D., Aden, A., Dudgeon, D. (2011). Process Design and Economics for Biochemical Conversion of Lignocellulosic Biomass to Ethanol Process Design and Economics for Biochemical Conversion of Lignocellulosic Biomass to Ethanol. Renewable Energy. Retrieved from http://www.nrel.gov/biomass/pdfs/47764.pdf), on the economics of lignocellulose hydrolysis, where production of cellulases is considered in some detail. Here the authors have consulted with experts in the field and produced this peer reviewed document which depicts 300 m³ production tanks seeded by a “seed train” of no less than three seed fermenters of 0.3, 3, and 30 m³. In a scenario without these seed tanks, the costs of design, purchase, piping, instrumentation, control, verification for insurance, maintenance running and operating costs can be completely avoided, with clear commercial implications.

Thus traditional knowledge has the consequence that the industry typically has built fermentation plants comprising seed fermenters and auxiliary equipment to runs such seed fermenters, such as supply tanks, means for stirring, controls for the seed fermenters such as pH, oxygen controls, which represent a significant cost both in establishment and operation of a large scale fermentation facility.

Consequently, will a fermentation facility intended to operate using the method according to the invention be established at a significantly lower cost compared with a traditional plant comprising seed tanks etc.

Further, the traditional way of operating a large scale fermentation typically starts with removing the microorganism for fermentation from a long time storage, such retrieving a vial from a freezer, inoculating the microorganism on an agar plate, when the microorganism has grown typically 24-96 hours, using microorganisms from the agar plate to inoculate the first seed fermenter, grow the microorganism typically 24-96 hours in the first seed fermenter, optionally using the ferment from the first seed fermenter to inoculate a second seed fermenter etc., until the main fermenter is inoculated with the broth from the last seed fermenter and the main fermentation begins. This means that the time from when it has been decided to use a particular microorganism in a fermentation process and until the main fermentation is complete and the product can be recovered, purified, formulated and delivered to the intended use or customer is long, which again reduces the flexibility of the fermentation facility since you need to know long time before use or delivery of a product, which product you need.

Using the method according to the invention will reduce the time from deciding to use a particular microorganism for production until the product is ready to use or deliver considerably compared with the traditional method involving seed fermenters because the time for handling and growing microorganism in the seed fermenters can be omitted. This provides for a significantly higher flexibility for a fermentation plant operating according to the invention because the time from deciding to produce a particular product until the product is ready can be reduced significantly. This is in particular an advantage for a multi purpose plant used for producing a range of different products by fermenting a range of different microorganisms each capable of expressing an intended product.

The fermentation process may according to the invention be of any known set-up, such as a batch process, a fed-batch process or a continuous fermentation process.

It is understood that a batch fermentation is a process where the growth medium is provided from the start, where the fermenter inoculated with the intended microorganism and the fermentation process is running until a predetermined condition have been reached, typically depletion of the growth medium and the cessation of microbial growth caused by the depletion.

A fed-batch process is a fermentation where a part of the growth medium is provided from the start of the fermentation process where the inoculum is added, and at a certain time point after the start of the fermentation additional substrate, feed, is fed to the fermenter at a rate that may be predetermined or determined by the conditions in the fermenter; until the maximal 3 volume has been reached. The feed may or may not have the same composition as the initial growth medium.

A continuous fermentation process is a process where new growth medium is continuously led to the fermenter and ferment is simultaneously removed from the fermenter at the same rate so the volume in the fermenter is constant.

The volume of the fermenter may be selected taking different factors into considerations such as type of fermentation, need for product, available equipment, oxygen requirement and capabilities of the equipment etc. This is all within the skills of the average practitioner. The main fermenter may have a volume from a few liters up to several thousand liters, e.g. 50 liters, 100 liters, 200 liters, 500 liters, 1000 liters 5000 liters, 10,000 liters, 50,000 liters, 100.000 liters or even more.

The microorganisms may in principle be any kind of cells such as mammalian cell lines, plant cells, insect cells, bacteria, and fungi. The microorganism may be a wild-type organism, i.e. a microorganism that is essentially unaltered from the organism that originally was isolated from nature, or may be a modified microorganism, that has been modified by one or more technical procedures such as mutagenesis or polyploidization using chemical or physical agents, genetic manipulations such as insertion or deletion of one or more genes using recombinant DNA technologies.

In one aspect of the invention, the microorganism is a bacterium. Examples of bacteria suitable for the present invention include the ones selected from the group comprising gram positive bacteria such as a Bacillus, Clostridium, Enterococcus, Geobacillus, Lactobacillus, Lactococcus, Oceanobacillus, Staphylococcus, Streptococcus, or Streptomyces, or a Gram-negative bacteria such as a Campylobacter, Escherichia, Flavobacterium, Fusobacterium, Helicobacter, Ilyobacter, Neisseria, Pseudomonas, Salmonella, or Ureaplasma.

In one aspect, the bacterial is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis.

In another aspect, the bacterium is a Streptococcus equisimilis, Streptococcus pyogenes, Streptococcus uberis, or Streptococcus equi subspecies Zooepidemicus.

In another aspect, the bacterium is a Streptomyces murinus, Streptomyces achromogenes, Streptomyces avermitilis, Streptomyces coelicolor, Streptomyces griseus, or Streptomyces lividans strain. In another aspect, the bacterial host cell is Escherichia coli.

In another aspect, the bacterium is selected from the group consisting of Bacillus, Streptomyces, Escherichia, Buttiauxella and Pseudomonas.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The microorganism may be a filamentous fungal strain such as an Acremonium, Agaricus, Alternaria, Aspergillus, Aureobasidium, Botryosphaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria strain.

In another aspect, the strain is an Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride strain.

In one aspect, the fungus is a strain selected from the group consisting of Candida, Hansenula, Kluyveromyces, Pichia, Saccharomyces, Schizosaccharomyces, Yarrowia, Acremonium, Aspergillus, Fusarium, Humicola, Mucor, Myceliophthora, Neurospora, Penicillium, Thielavia, Tolypocladium, and Trichoderma.

In a more preferred embodiment, the filamentous fungus is selected from the group consisting of Trichoderma and Aspergillus host cells, in particular a strain of Trichoderma harzianum, Trichoderma koningii, Trichoderma longibrachiatum, Trichoderma reesei, Trichoderma viridel, Aspergillus awamori, Aspergillus fumigatus, Aspergillus foetidus, Aspergillus japonicus, Aspergillus nidulans, Aspergillus niger or Aspergillus oryzae, especially a strain of Trichoderma reesei.

The microorganism may be a yeast cell such as a Candida, Hansenula, Kluyveromyces, Pichia, Saccharomyces, Schizosaccharomyces, or Yarrowia strain. In another aspect, the strain is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis strain.

The fermentation product may in principle by any fermentation product, such as a protein of interest, a primary metabolite such as citric acid, malic acid or succinic acid; or a secondary metabolite such as penicillin, bacitracin, cephalosporin or clavulanic acid. Preferably the fermentation product is a protein.

The protein of interest may in principle be any protein that can be produced in a fermentation process, but according to the invention the protein of interest is preferably an enzyme. Examples of enzymes includes hydrolases, oxidases, isomerases, e.g. amylase, alpha-amylase, glucoamylase, pullulanase, protease, metalloprotease, peptidase, lipase, cutinase, acyl transferase, cellulase, endoglucanase, glucosidase, cellubiohydrolase, xylanase, xyloglucantransferase, xylosidase, mannanase, phytase, phosphatase, xylose isomerase, glucoase isomerase, lactase, acetolactate decarboxylase, pectinase, pectin methylesterase, polygalacturonidase, lyase, pectate lyase, arabinase, arabinofuranosidase, galactanase, a laccase, peroxidase and an asparaginase.

According to the invention the fermenter is inoculated directly without the use of a seed fermenter. The inoculum material, i.e. the microorganism may in principle be in any viable form. Thus the inoculum may be in form of a vial removed from the freezer, an ampoule containing the inoculum material, an agar plate containing the microorganism or a shake flask containing a culture of the microorganism.

In one embodiment the inoculum is derived from an agar plate where the microorganism has grown. The agar plate and the microorganism is transferred to a sterile small bowl and mixed with sterile water or an aqueous solution comprising salts, surfactants and/or buffers, and transfer the whole to the fermenter. In another embodiment the inoculum is derived from a vial. The vial is removed from freezer, thawed and transferred to a sterile small bowl and mixed with sterile water or an aqueous solution comprising salts, surfactants and/or buffers, and transferred to the fermenter. After inoculation the microorganism is fermented in the fermenter using similar conditions as usually are applied for fermentations using a seed fermenter.

The inoculum may be transferred to the main fermenter using any suitable sterile technique. One particular preferred method is transferring the microorganism to a sterile bowl using sterile water and thereafter transferring the mixture of the microorganism and sterile water to the main fermenter.

When fermentation is complete the protein product may be recovered and/or purified using down stream processes well known in the art.

### EXAMPLES

### Materials and Methods

### Fermentation Medium:

| | | |
|---|---|---|
| KH₂PO₄ | 40 | g |
| (NH₄)₂SO₄ | 63.5 | g |
| MgSO₄•7H₂O | 20 | g |
| CaCl₂•6H₂O | 7.4 | g |
| Glucose•H₂O | 500 | g |
| Corn Steep powder | 62.5 | g |
| FeSO₄•7H₂O | 0.05 | g |
| MnSO₄•H₂O | 0.016 | g |
| ZnSO₄•7H₂O | 0.014 | g |
| Dowfax ® 63N10 | 5 | ml |
| Water to 10 kg | | |

Dowfax® 63N10 is Nonionic Surfactant used as defoaming agent. It is a linear ethylene oxide/propylene oxide block polymer provided by The DOW Chemical Company, (Middelsex, United Kingdom).

### Fermenter

The fermenter used in the examples was a standard lab scale (20 l) fermenter.

### Enzyme Assays

Total protein was measured using the Pierce™ BCA Protein Assay Kit (ThermoFisher scientific cat. nr. 23227, provided by Life Technologies Europe BV; Naerum, Denmark) according to the manufacturer's instructions.

Beta-xylosidase activity can be determined using 1 mM p-nitrophenyl-beta-D-xyloside as substrate in 100 mM sodium citrate containing 0.01% TWEEN® 20 at pH 5, 40° C. One unit of beta-xylosidase is defined as 1.0 μmole of p-nitrophenolate anion produced per minute at 40° C., pH 5 from 1 mM p-nitrophenyl-beta-D-xyloside in 100 mM sodium citrate containing 0.01% TWEEN® 20.

### Example 1 Seed Fermenter

A 20 l fermenter comprising 10 kg of the medium described above was sterilized by heating one hour at 123° C. After cooling to 25° C. pH was adjusted to 5.0 using H₃PO₄ and/or NH₃.

An PDA agar plate comprising a Trichoderma reesei strain that had grown for 7 days at 30° C. was used of inoculation. The microorganism was transferred to a sterile bowl approximately 25 ml water and the whole mixture was inoculated into the fermenter.

The fermenter was run for 45 hours at 28° C. at an oxygen saturation of approximately 40%, whereafter the culture is ready for inoculation into the main fermenter.

### Example 2 Main Fermenter, Inoculation from Seed Tank

A 20 l fermenter comprising 10 kg of the medium described above was sterilized by heating one hour at 123° C. After cooling to 25° C. pH was adjusted to 5.0 using H₃PO₄ and/or NH₃.

The fermenter was inoculated with 1000 g of the culture prepared in Example 1 and fermentation was started at a temperature of 28° C. and at an oxygen saturation of approximately 40%.

After 30 minutes 20% lactose was fed to the fermenter controlled by the oxygen saturation of 40%:

Between 0.5 and 50 hours lactose in amounts between 20 g/hour and 130 g/hour was fed, and between 50 and 100 hours between 20 g/hour and 240 g/hour was fed into the fermenter. The fermenter was run for 191 hours in total.

Samples was taken at regular intervals and analysed for total protein and for beta-xylosidase activity.

The fermentation was repeated.

### Example 3 Main Fermenter, Inoculation from Vial

A 20 l fermenter comprising 10 kg of the medium described above was sterilized by heating one hour at 123° C. After cooling to 25° C. pH was adjusted to 5.0 using H₃PO₄ and/or NH₃.

The fermenter was inoculated with a vial with the same Trichoderma reesei strain as used in Example 1. The vial was removed from the freezer, thawed and the culture transferred to the fermenter with approximately 10 ml sterile water. Fermentation was started at a temperature of 28° C. and at an oxygen saturation of approximately 40%.

After 30 minutes 20% lactose was fed to the fermenter controlled by the oxygen saturation of 40%:

Between 0.5 and 50 hours lactose in amounts between 20 g/hour and 130 g/hour was fed, and between 50 and 100 hours between 20 g/hour and 240 g/hour was fed into the fermenter. The fermenter was run for 191 hours in total.

Samples was taken at regular intervals and analysed for total protein and for beta-xylosidase activity.

### Example 4 Main Fermenter, Inoculation from Agar

A 20 l fermenter comprising 10 kg of the medium described above was sterilized by heating one hour at 123° C. After cooling to 25° C. pH was adjusted to 5.0 using H₃PO₄ and/or NH₃.

The fermenter was inoculated with a vial with the same microorganism as used in example 1. An PDA agar plate comprising the Trichoderma reesei strain that had grown for 7 days at 30° C. was used of inoculation. The microorganism was transferred to a sterile bowl approximately 25 ml water and the whole mixture was inoculated into the fermenter. Fermentation was started at a temperature of 28° C. and at an oxygen saturation of approximately 40%.

After 30 minutes 20% lactose was fed to the fermenter controlled by the oxygen saturation of 40%:

Between 0.5 and 50 hours lactose in amounts between 20 g/hour and 130 g/hour was fed, and between 50 and 100 hours between 20 g/hour and 240 g/hour was fed into the fermenter. The fermenter was run for 191 hours in total.

Samples was taken at regular intervals and analysed for total protein and for beta-xylosidase activity.

### Example 5 Analysis

Samples from example were analysed for total protein and beta-xylosidase and normalized according to the highest value achieved in the references. Following results were obtained

| | Time (hours) | Total protein | beta-xylosidase |
|---|---|---|---|
| Inoculation from vial | 44 | 13.24% | 0.54% |
| | 68 | 15.16% | 3.92% |
| | 92 | 35.89% | 21.58% |
| | 116 | 56.97% | 36.83% |
| | 140 | 67.60% | 47.92% |
| | 164 | 78.57% | 61.19% |
| | 191 | 73.34% | 54.65% |
| Inoculation from Agar | 44 | 13.07% | 4.79% |
| | 68 | 25.26% | 12.57% |
| | 92 | 44.95% | 35.45% |
| | 116 | 65.68% | 60.10% |
| | 140 | 81.88% | 87.80% |
| | 164 | 94.60% | 89.31% |
| | 191 | 88.15% | 82.87% |
| Reference 1 Seed tank | 44 | 28.75% | 20.69% |
| | 68 | 44.43% | 34.26% |
| | 92 | 54.18% | 50.89% |
| | 116 | 80.66% | 79.50% |
| | 140 | 94.43% | 97.23% |
| | 164 | 87.46% | 51.19% |
| | 191 | 82.23% | 78.32% |
| Reference 2 seed tank | 42 | 27.00% | 23.17% |
| | 66 | 42.33% | 37.23% |
| | 90 | 63.07% | 60.20% |
| | 114 | 78.05% | 79.21% |
| | 138 | 90.59% | 94.06% |
| | 162 | 100.00% | 100.00% |
| | 189 | 84.84% | 81.19% |

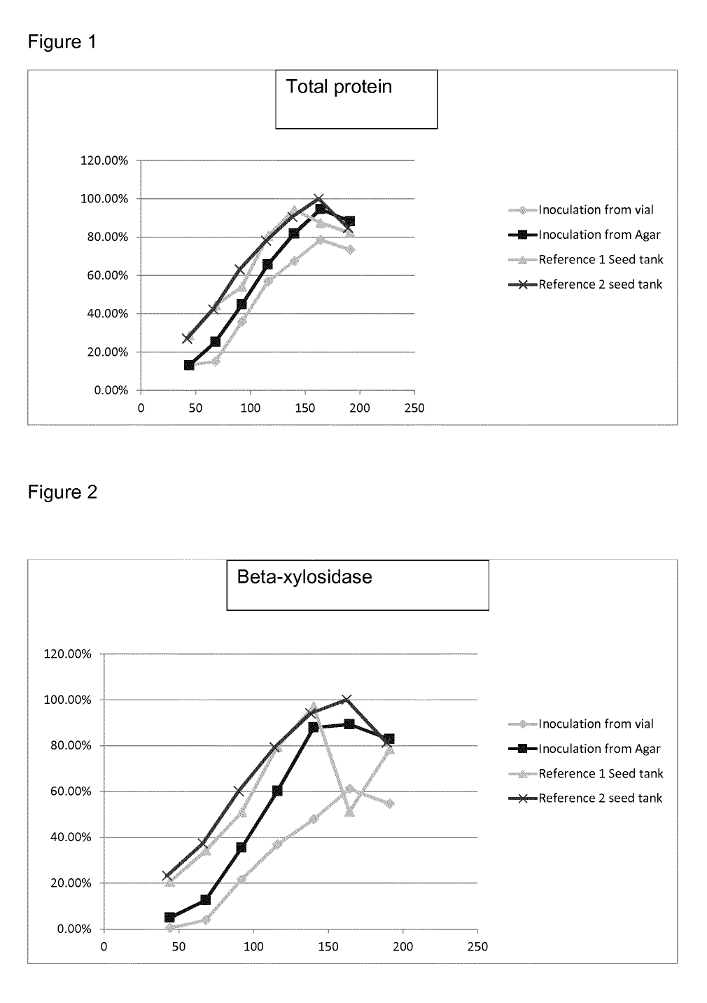

Results are also shown in FIGS. 1 and 2.
Time from start (i.e. the time the vial was removed from freezer) in the four fermentations are:

| | agar | seed | Main | Total |
|---|---|---|---|---|
| | plate growth | fermentation | fermentation | time |
| | (hours) | (Hours) | (Hours) | (Hours) |
| Reference 1 | 168 (7 days) | 45 | 191 | 404 |
| Seed tank | | | | |
| Reference 2 | 168 (7 days) | 45 | 189 | 402 |
| Seed tank | | | | |
| From vial | 0 | 0 | 191 | 191 |
| From agar | 168 (7 days) | 0 | 191 | 359 |

The results show that the fermentations inoculated from agar and from a vial gave yields that was slightly lower that the reference, however, the time for the process is also significantly reduced using the method according to the invention.

### Example 6

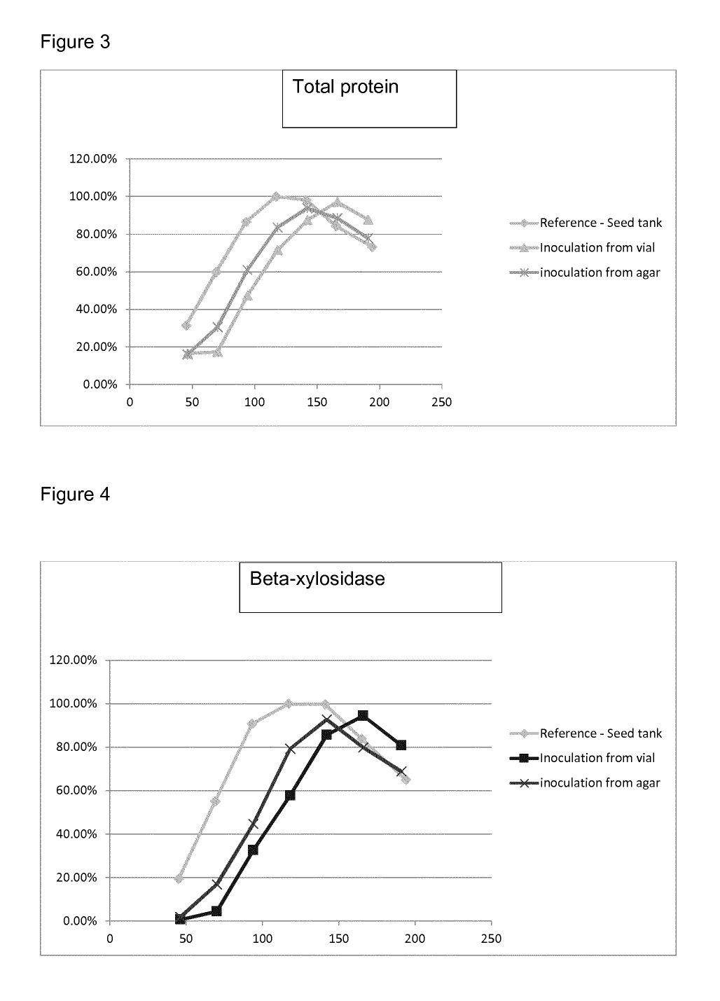

The experiments disclosed in examples 1-4 was repeated with only one reference fermentation and the data analysed as described in Example 5. The results disclosed in the table below and in FIG. 3 or 4.

| | Time (hours) | Total Protein | Beta-xylosidase |
|---|---|---|---|
| Reference - Seed tank | 45 | 31.49% | 19.43% |
| | 69 | 59.73% | 55.13% |
| | 93 | 86.64% | 90.70% |
| | 117 | 100.00% | 100.00% |
| | 141 | 98.09% | 99.59% |
| | 165 | 84.35% | 83.86% |
| | 194 | 73.28% | 64.98% |
| Inoculation from vial | 46 | 16.60% | 0.62% |
| | 70 | 17.37% | 4.39% |
| | 94 | 47.33% | 32.56% |
| | 118 | 71.56% | 57.73% |
| | 142 | 87.40% | 85.64% |
| | 166 | 97.14% | 94.39% |
| | 191 | 87.60% | 80.71% |
| inoculation from agar | 46 | 16.03% | 1.78% |
| | 70 | 30.53% | 16.83% |
| | 94 | 61.07% | 44.73% |
| | 118 | 83.59% | 79.21% |
| | 142 | 93.70% | 92.75% |
| | 166 | 88.55% | 79.75% |
| | 191 | 77.86% | 68.81% |

Time from start (i.e. the time the vial was removed from freezer) in the four fermentations are:

| | agar | seed | Main | Total |
|---|---|---|---|---|
| | plate growth | fermentation | fermentation | time |
| | (hours) | (Hours) | (Hours) | (Hours) |
| Reference | 168 (7 days) | 45 | 194 | 407 |
| Seed tank | | | | |
| From vial | 0 | 0 | 191 | 191 |
| From agar | 168 (7 days) | 0 | 191 | 359 |

The results confirms the conclusions from example 1-5 that by inoculating the main fermenter directly from agar plate or from a vial taken directly from the freezer provides for a production of the desired protein product in a comparable yield but in a significantly shorter time.

The small differences that are seen between the two set of experiments are only batch to batch variations that frequently seen in the area.

1. A method for fermenting a microorganism producing a protein product, comprising inoculating a fermenter with said microorganism and fermenting said microorganism, wherein the inoculating is done directly with the microorganism without using a seed fermenter.

2. The method of claim 1, wherein the fermenting is a submerged fermentation process.

3. The method of claim 2, wherein the fermentation process is a batch fermentation, a fed-batch fermentation or a continuous fermentation.

4. The method of claim 1, wherein the microorganism is selected from bacteria, fungi, yeast, insect cells, mammalian cells or plant cells.

5. The method of claim 4, wherein the microorganism is a recombinant organism.

6. The method of claim 4, wherein the microorganism is a bacterium selected from the group consisting of Gram-positive bacteria and Gram-negative bacteria.

7. The method of claim 6, wherein the Gram-positive bacteria are selected from the group consisting of Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis.

8. The method of claim 4 wherein the microorganism is a filamentous fungus or a yeast.

9. The method of claim 8, wherein the filamentous fungus is selected from the group consisting of Acremonium, Agaricus, Alternaria, Aspergillus, Aureobasidium, Botryosphaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, and Xylaria strain.

10. The method of claim 9, wherein the filamentous fungus is selected from the group consisting of Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, and Trichoderma viride.

11. The method of claim 8, wherein the yeast is selected from the group consisting of Candida, Hansenula, Kluyveromyces, Pichia, Saccharomyces, Schizosaccharomyces, and Yarrowia.

12. The method of claim 1 wherein the protein product is an enzyme.

13. The method of claim 12, wherein the enzyme is selected from the group consisting of hydrolases, oxidases, and isomerases, including amylase, alpha-amylase, glucoamylase, pullulanase, protease, metalloprotease, peptidase, lipase, cutinase, acyl transferase, cellulase, endoglucanase, glucosidase, cellubiohydrolase, xylanase, xyloglucantransferase, xylosidase, mannanase, phytase, phosphatase, xylose isomerase, glucoase isomerase, lactase, acetolactate decarboxylase, pectinase, pectin methylesterase, polygalacturonidase, lyase, pectate lyase, arabinase, arabinofuranosidase, galactanase, laccase, peroxidase and asparaginase.

14. The method of claim 1, wherein the fermenter has a volume of more than 50 litre.

15. The method of claim 1, wherein the inoculating uses a microorganism from an agar plate, freeze dried culture or frozen culture.

16. The method of claim 6, wherein the Gram-positive bacteria are selected from the group consisting of Bacillus, Clostridium, Enterococcus, Geobacillus, Lactobacillus, Lactococcus, Oceanobacillus, Staphylococcus, Streptococcus, and Streptomyces.

17. The method of claim 6, wherein the Gram-negative bacteria are selected from the group consisting of Campylobacter, Escherichia, Flavobacterium, Fusobacterium, Helicobacter, Ilyobacter, Neisseria, Pseudomonas, Salmonella, and Ureaplasma.

18. The method of claim 8, wherein the yeast are selected from the group consisting of Saccharomyces carlsbergensis, Saccharomyces cerevisiae, Saccharomyces diastaticus, Saccharomyces douglasii, Saccharomyces kluyveri, Saccharomyces norbensis, and Saccharomyces oviformis.

19. A method, comprising:
inoculating a main fermenter of at least 20 liter volume, directly with a microorganism from an agar plate culture, frozen culture or freeze-dried culture; and
fermenting the microorganism in the main fermenter to an exponential or stationary growth phase;
wherein an amount of total protein in the main fermenter after the fermenting is equal to or greater than an amount of total protein in a main fermenter that is inoculated with the microorganism from a seed fermenter and fermented to an equivalent growth phase; and
wherein a total time for production for the direct-inoculation process is the same as or less than a total time for production for the seed fermenter-process.
